(19) United States
(12) Patent Application Publication
(10) Pub. No.: US 2017/0049406 A1
(43) Pub. Date: Feb. 23, 2017

(54) SIMULTANEOUS DISPLAY OF USER LOCATION AND PHYSIOLOGICAL DATA

(71) Applicant: COVIDIEN LP, Mansfield, MA (US)

(72) Inventors: AARON JOHN LANZEL, Annapolis, MD (US); BENJAMIN DAVID MORRIS, Annapolis, MD (US); ROBERT JEFFREY NELSEN, Millersville, MD (US)

(21) Appl. No.: 15/239,453

(22) Filed: Aug. 17, 2016

Related U.S. Application Data
(60) Provisional application No. 62/206,635, filed on Aug. 18, 2015.

(51) Int. Cl. A61B 5/00; G06T 11/20; G06T 11/60; A61B 5/0205

(52) U.S. Cl. CPC A61B 5/7425; A61B 5/02055; G06T 11/206; G06T 11/60; A61B 5/02438

(57) ABSTRACT

Methods, apparatuses and systems are described for simultaneously displaying location data and physiological data for a plurality of users. The methods may include receiving physiological data corresponding to one or more physiological parameters of each of the plurality of users. The methods may also include receiving location data corresponding to a location of each of the plurality of users. The method may also include displaying the received physiological data and location data simultaneously on a same visual representation for each of the plurality of users.

### CROSS REFERENCE

The present application for patent claims priority to U.S. Provisional Patent Application No. 62/206,635 by Lanzel et al., entitled “Simultaneous Display of User Location and Physiological Data,” filed Aug. 18, 2015, assigned to the assignee hereof.

### BACKGROUND

The present disclosure relates generally to physiological monitoring systems, and more particularly to displaying physiological data and location data for a plurality of users simultaneously on a same visual representation.

Use of mobile personal monitoring devices in sports and physical activity applications is well known, but many of these activity monitors may be limited in their functionality with respect to quantifying and visualizing training parameters. For example, many activity monitoring systems may have limited display capabilities, making compilation and comparison of multiple sets of data for a single user, and moreover, comparison of data sets for multiple users, more difficult to visualize or contextualize.

Existing performance monitoring systems may enable the capture and transmission of various physiological data for a user via mobile and fixed data networks, to enable remote monitoring of user performance and physiological conditions. Monitored physiological data may include heart rate, R-R interval, breathing rate, posture, activity level, peak acceleration, speed and distance, GPS, step count, and the like. Existing performance monitoring programs may be limited, however, in the scope of their display functionalities. For example, a plurality of players' physiological data may be displayed as a list, graph, or series of numerical data, but may not be viewable in a more relatable, real-world context.

Similarly, existing global positioning systems may provide visual indicators of a user's position in an area over time, but may not be operable to illustrate physiological parameters associated with the user. Accordingly, a person interested in monitoring one or more users' positions and physiological parameters may be required to cross-reference information displayed separately on a map and a graph or table, which may be inconvenient and inefficient.

### SUMMARY

For sports and other physical activity monitoring, it may be beneficial to view user position and physiological data concurrently on the same display, in a manner that is easily understood and analyzed. In particular, it may be beneficial to view user physiological data overlaid on user position data on a map or other positional image, such that the relative position and physiological state for a user over time, or for a plurality of users at the same point in time or over a period of time, may be readily understood. One method of accomplishing this goal may include displaying a user's position as a point or dot, line, or trail on a map, or on an image of a sports field or other location. The point, line, or trail representing the user's position may be displayed using any combination of colors, opacity, width, shape, or the like, in order to indicate associated physiological data parameters. For example, the color red may be indicative of a heart rate above 90% of the user's maximum heart rate, such that the user's current position and heart rate may be readily understood from viewing a map showing a red point or line. The user's position and physiological data may be updated in real-time or at predetermined intervals, such that the point indicating the user's position, and the color of that point indicating, for example, the user's heart rate as a percentage of his maximum heart rate, may be similarly continuously updated to show current user data.

Although described with respect to heart rate, any other physiological and physical parameters may be monitored and displayed on the map or image, including speed, altitude, distance, respiration rate, heart rate variability, blood oxygen levels, and the like. In some examples, two or more physiological parameters may be displayed concurrently. For example, a shape of the point on the map indicating the user's position, such as a circle, square, star, triangle, etc., may indicate the user's heart rate, while the color of the point may indicate the user's respiration rate. In other examples, the size of the point indicating the user's position on the map may be indicative of a physiological parameter; for example, a smaller point may indicate a slower speed, while a larger point may indicate a faster speed. In still other examples, the “heat glow” of a point may represent a physiological parameter of the user. For example, a point having a larger glow area—an area of glowing color—may indicate a high respiration rate, while a point having a smaller glow area may indicate a low respiration rate.

The present disclosure is accordingly directed to a method for simultaneously displaying location data and physiological data for a plurality of users. In some embodiments, the method may include: receiving physiological data corresponding to one or more physiological parameters of each of the plurality of users; receiving location data corresponding to a location of each of the plurality of users; and displaying the received physiological data and location data simultaneously on a same visual representation for each of the plurality of users.

In some embodiments, the method may further include displaying the physiological data and the location data on the same visual representation on a map or image.

In some embodiments, the method may further include indicating the physiological data and the location data on the same visual representation through a combination of two or more of a dot, a line, a color, a heat radius, or a shape.

In some embodiments, the method may further include continuously updating at least a portion of the visual representation for each of the plurality of users based at least in part on one or more of received physiological data and location data. In some embodiments, continuously updating at least a portion of the visual representation may include altering a shape of the visual representation, altering a color of the visual representation, altering a heat radius of the visual representation, altering a position of the visual representation, or altering an opacity of the visual representation, or a combination thereof.

In some embodiments, displaying the received physiological and location data may include displaying the received physiological data and location data in real-time.

In some embodiments, displaying the received physiological data and location may include displaying the received physiological data and location data for a predetermined period of time. In some embodiments, at least one of the received physiological data and location data may be displayed as an average over the predetermined period of time.

In some embodiments, the one or more physiological parameters of the plurality of users may include a heart rate, a respiration rate, a body temperature, a mechanical intensity, a physiological intensity, a training intensity, a speed, a distance traveled, a time spent in a position, or an altitude, or a combination thereof.

In some embodiments, the method may further include associating the received physiological data with a plurality of predetermined training zones. In some embodiments, each of the plurality of predetermined training zones may be associated with a respective shape, size, opacity, heat radius, or color. In some embodiments, the plurality of predetermined training zones may be determined based at least in part on individual physiological parameters of each of the plurality of users, or individual training goals of each of the plurality of users, or a combination thereof.

The present disclosure is also directed to a system for simultaneously displaying location data and physiological data for a plurality of users. In some embodiments, the system may include: a transceiver configured to receive physiological data corresponding to one or more physiological parameters of each of the plurality of users and location data corresponding to a location of each of the plurality of users from one or more sensors; and a processor configured to simultaneously display the received physiological data and location data on a same visual representation for each of the plurality of users.

The present disclosure is also directed to a non-transitory computer-readable medium storing computer-executable code. In some embodiments, the code may be executable by a processor to: receive physiological data corresponding to one or more physiological parameters of each of a plurality of users; receive location data corresponding to a location of each of the plurality of users; and display the received physiological data and location data simultaneously on a same visual representation for each of the plurality of users.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Further scope of the applicability of the described methods and apparatuses will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 is a block diagram of an example of a physiological parameter and user location monitoring system in accordance with various embodiments;

FIGS. 2A and 2B are example user interfaces displaying physiological data and location data on a same visual representation, in accordance with various embodiments;

FIGS. 3A and 3B are example illustrations of images displaying physiological data and location data on a same visual representation, in accordance with various embodiments;

FIG. 4 is a block diagram of an example of an apparatus in accordance with various embodiments;

FIG. 5 is a block diagram of an example of an apparatus in accordance with various embodiments;

FIG. 6 is a block diagram of an example of an apparatus in accordance with various embodiments;

FIG. 7 is a block diagram of an example of a server for facilitating simultaneous display of user location and physiological data in accordance with various embodiments; and

FIG. 8 is a flowchart of a method for simultaneously displaying user location and physiological data, in accordance with various embodiments.

### DETAILED DESCRIPTION

In order to easily track one or more user's location and physiological data simultaneously, it may be desirable to display the one or more user's location and physiological data using a single visual representation for each user. A visual representation may be preferable over graphical or numerical displays, the latter of which may be more cumbersome in conveying pertinent physiological or training data. A user wishing to track his own location and physiological data, or a healthcare provider or sports trainer wishing to track location and physiological data for his subject, may be interested in a variety of physiological parameters, viewable in relation to the user's real-time position. These parameters may include the user's heart rate, blood pressure, oxygen saturation levels, glucose levels, etc. Additionally, physical parameters such as speed, distance, elevation, and the like, may be of interest. Consolidating and presenting these physiological parameters to the user or his coach or clinician in a succinct, easy-to-read form, therefore, may be particularly valuable.

For example, a user may wear one or more monitors, such as a chest strap, pod, or wrist-worn monitor having integrated or associated sensors configured to detect location data or physiological parameters, or a combination thereof. The wearable sensors may collect location data and/or physiological parameters from the user on an ongoing basis, or at predetermined intervals, and may either process the collected data locally, or may communicate the data to a local or remote computing device or network for processing. The collected location data and/or physiological parameters may then be consolidated into a single visual representation, for example in the form of colored points or lines on a map or image.

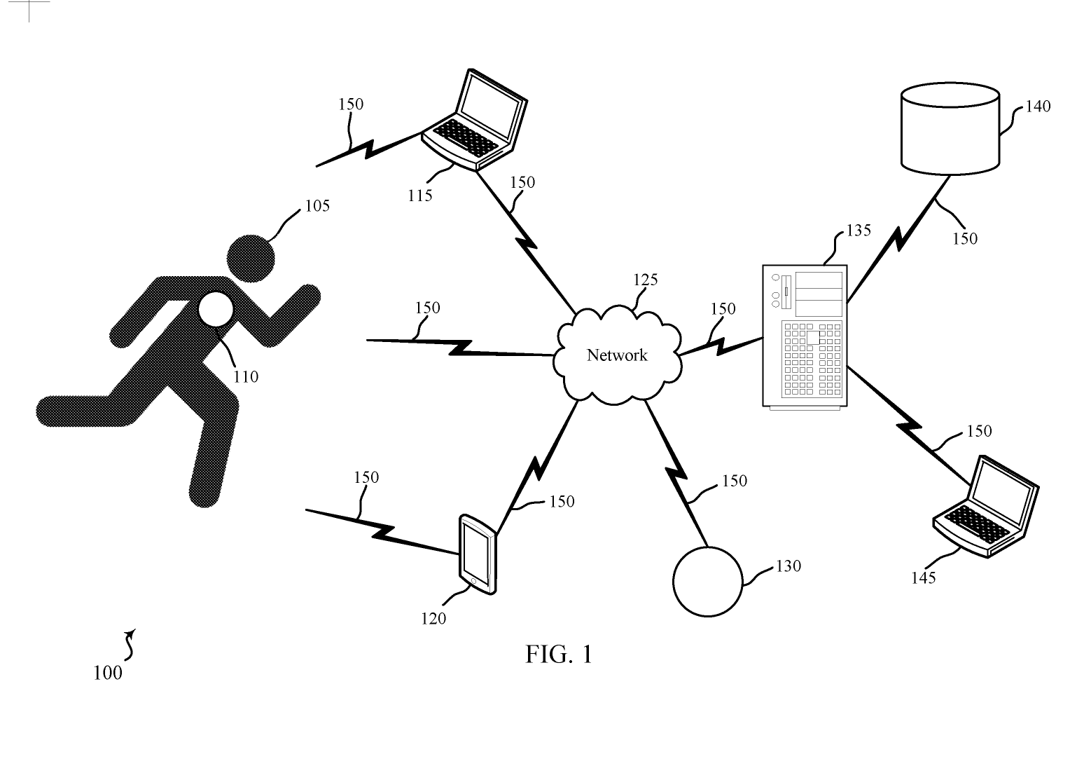

Referring first to FIG. 1, a diagram illustrates an example of a location and physiological parameter monitoring system 100. The system 100 includes user 105, wearing or carrying one or more sensor unit 110. The user 105 may be an athlete in some examples, may be a patient in other examples, or in some instances may be a layperson interested in simply monitoring various aspects of his or her daily activities. The sensor units 110 may transmit signals via wireless communication links 150. The transmitted signals may be transmitted to local computing devices 115, 120. Local computing device 115 may be a local caregiver's station or a personal computing device monitored by a coach, for example. Local computing device 120 may be a mobile device, for example. The local computing devices 115, 120 may be in communication with a server 135 via network 125. The sensor units 110 may also communicate directly with the server 135 via the network 125. Additional, third-party sensors 130 may also communicate directly with the server 135 via the network 125. The server 135 may be in further communication with a remote computing device 145, thus allowing a caregiver to remotely monitor the user 105. The server 135 may also be in communication with various remote databases 140 where the collected data may be stored.

The sensor units 110 are described in greater detail below. Each sensor unit 110 is capable of sensing multiple location and physiological parameters. Thus, the sensor units 110 may each include multiple sensors such as heart rate and ECG sensors, respiratory rate sensors, accelerometers, and global positioning sensors. For example, a first sensor in a sensor unit 110 may be an oxygen saturation monitor or a glucose level monitor operable to detect a user's blood oxygen or sugar levels. A second sensor within a sensor unit 110 may be operable to detect a second physiological parameter. For example, the second sensor may be a heart rate monitor, an electrocardiogram (ECG) sensing module, a breathing rate sensing module, and/or any other suitable module for monitoring any suitable physiological parameter. A third sensor in sensor unit 110 may be a global positioning sensor operable to monitor the user's location in real-time. Multiple sensor units 110 may be used on a single user 105. The sensor units 110 may be worn or carried by the user 105 through any known means, for example as a wearable chest strap or wristwatch-type device, or the like. In other examples, the sensor units 110 may be integrated with the user's clothing. The data collected by the sensor units 110 may be wirelessly conveyed to either the local computing devices 115, 120 or to the remote computing device 145 (via the network 125 and server 135). Data transmission may occur via, for example, frequencies appropriate for a personal area network (such as Bluetooth or IR communications) or local or wide area network frequencies such as radio frequencies specified by the IEEE 802.15.4 standard.

Each data point recorded by the sensor units 110 may include an indication of the time the measurement was made (referred to herein as a “timestamp”). In some embodiments, the sensor units 110 are sensors configured to conduct periodic automatic measurements of one or more location or physiological parameters. A user may wear or otherwise be attached to one or more sensor units 110 so that the sensor units 110 may measure, record, and/or report location and physiological data associated with the user.

The sensor units 110 may be discrete sensors, each having independent clocks. As a result, sensor units 110 may generate data with different frequencies. The data streams generated by the sensor units 110 may also be offset from each other. The sensor units 110 may each generate a data point at any suitable time interval.

The local computing devices 115, 120 may enable the user 105 and/or a local caregiver or coach to monitor the collected user location and physiological data. For example, the local computing devices 115, 120 may be operable to present data collected from sensor units 110 in a human-readable format. For example, the received data may be outputted as a display on a computer or a mobile device. The local computing devices 115, 120 may include a processor that may be operable to present data received from the sensor units 110 in a visual format. In some examples, the location and physiological data may be displayed simultaneously on a single visual display, such as a map or other image. The local computing devices 115, 120 may also output data in an audible format using, for example, a speaker.

The local computing devices 115, 120 may be custom computing entities configured to interact with the sensor units 110. In some embodiments, the local computing devices 115, 120 and the sensor units 110 may be portions of a single sensing unit operable to sense and display physiological parameters, for example on a wrist-worn monitor. In another embodiment, the local computing devices 115, 120 may be general purpose computing entities such as a personal computing device, for example, a desktop computer, a laptop computer, a netbook, a tablet personal computer (PC), an iPod®, an iPad®, a smartphone (e.g., an iPhone®, an Android® phone, a Blackberry®, a Windows® phone, etc.), a mobile phone, a personal digital assistant (PDA), and/or any other suitable device operable to send and receive signals, store and retrieve data, and/or execute modules.

The local computing devices 115, 120 may include memory, a processor, an output, a data input, and a communication module. The processor may be a general purpose processor, a Field Programmable Gate Array (FPGA), an Application Specific Integrated Circuit (ASIC), a Digital Signal Processor (DSP), and/or the like. The processor may be configured to retrieve data from and/or write data to the memory. The memory may be, for example, a random access memory (RAM), a memory buffer, a hard drive, a database, an erasable programmable read only memory (EPROM), an electrically erasable programmable read only memory (EEPROM), a read only memory (ROM), a flash memory, a hard disk, a floppy disk, cloud storage, and/or so forth. In some embodiments, the local computing devices 115, 120 may include one or more hardware-based modules (e.g., DSP, FPGA, ASIC) and/or software-based modules (e.g., a module of computer code stored at the memory and executed at the processor, a set of processor-readable instructions that may be stored at the memory and executed at the processor) associated with executing an application, such as, for example, receiving and displaying data from sensor units 110.

The data input module of the local computing devices 115, 120 may be used to manually input measured physiological and location data instead of or in addition to receiving data from the sensor units 110. For example, a third-party user of the local computing device 115, 120 may make an observation as to one or more physiological or location conditions of a monitored user and record the observation using the data input module. A third-party user may be, for example, a nurse, a doctor, a coach, and/or any other medical healthcare or physical training professional authorized to record user observations, the monitored user, and/or any other suitable user. For instance, the third-party user may measure the monitored user's body temperature (e.g., using a stand-alone thermometer) and enter the measurement into the data input module. In some embodiments, the data input module may be operable to allow the third-party user to select “body temperature” and input the observed temperature into the data input module, e.g., using a keyboard. The data input module may timestamp the observation (or measurement) with the time the observation is input into the local computing devices 115, 120, or the local computing devices 115, 120 may prompt the third-party user to input the time the observation (or measurement) was made so that the time provided by the third-party user is used to timestamp the data point. In another example, a third-party user may observe the current location of the user, for example on a sports field, and may input corresponding location observations into the local computing devices 115, 120.

The processor of the local computing devices 115, 120 may be operated to control operation of the output of the local computing devices 115, 120. The output may be a television, a liquid crystal display (LCD) monitor, a cathode ray tube (CRT) monitor, speaker, tactile output device, and/or the like. In some embodiments, the output may be an integral component of the local computing devices 115, 120. Similarly stated, the output may be directly coupled to the processor. For example, the output may be the integral display of a tablet and/or smartphone. In some embodiments, an output module may include, for example, a High Definition Multimedia Interface™ (HDMI) connector, a Video Graphics Array (VGA) connector, a Universal Serial Bus™ (USB) connector, a tip, ring, sleeve (TRS) connector, and/or any other suitable connector operable to couple the local computing devices 115, 120 to the output.

As described in additional detail herein, at least one of the sensor units 110 may be operable to transmit physiological and/or location data to the local computing devices 115, 120 and/or to the remote computing device 145 continuously, at scheduled intervals, when requested, and/or when certain conditions are satisfied (e.g., during an alarm condition).

The remote computing device 145 may be a computing entity operable to enable a remote user to monitor the output of the sensor units 110. The remote computing device 145 may be functionally and/or structurally similar to the local computing devices 115, 120 and may be operable to receive data streams from and/or send signals to at least one of the sensor units 110 via the network 125. The network 125 may be the Internet, an intranet, a personal area network, a local area network (LAN), a wide area network (WAN), a virtual network, a telecommunications network implemented as a wired network and/or wireless network, etc. The remote computing device 145 may receive and/or send signals over the network 125 via communication links 150 and server 135.

The remote computing device 145 may be used by, for example, a healthcare professional or sports coach to monitor the output of the sensor units 110. In some embodiments, as described in further detail herein, the remote computing device 145 may receive an indication of physiological and/or location data when the sensors detect an alert condition, when the healthcare provider or coach requests the information, at scheduled intervals, and/or at the request of the healthcare provider, coach, and/or the user 105. For example, the remote computing device 145 may be operable to receive summarized physiological and/or location data from the server 135 and display the summarized data in a convenient format. The convenient format may take the form of, for example, a line, point, or series of points on a map or image, where each line, point, or series of points corresponds to location and/or physiological data for each of one or more monitored users. The remote computing device 145 may be located, for example, at a nurses' station or in a user's room in some examples, or in other instances may be located at a personal computing device monitored by a coach or other professional monitoring the user, and may be configured to simultaneously display a visual representation of the physiological and location data collected from one or more users. In some instances, the local computing devices 115, 120 may also be operable to receive and display physiological and/or location data in much the same way that the remote computing device 145 is operable.

The server 135 may be configured to communicate with the sensor units 110, the local computing devices 115, 120, the third-party sensors 130, the remote computing device 145, and databases 140. The server 135 may perform additional processing on signals received from the sensor units 110, local computing devices 115, 120 or third-party sensors 130, or may simply forward the received information to the remote computing device 145 and databases 140. The databases 140 may be examples of electronic health records (“EHRs”) and/or personal health records (“PHRs”), and may be provided by various service providers. The third-party sensor 130 may be a sensor that is not attached to the user 105 but that still provides location and/or physiological data that may be useful in connection with the data provided by sensor units 110. In other examples, the third-party sensor 130 may be worn or carried by, or associated with, a third-party user, and data therefrom may be used for comparison purposes with data collected from the user 105. In certain embodiments, the server 135 may be combined with one or more of the local computing devices 115, 120 and/or the remote computing device 145.

The server 135 may be a computing device operable to receive data streams (e.g., from the sensor units 110 and/or the local computing devices 115, 120), store and/or process data, and/or transmit data and/or data summaries (e.g., to the remote computing device 145). For example, the server 135 may receive a stream of heart rate data from a sensor unit 110, a stream of oxygen saturation data from the same or a different sensor unit 110, and a stream of location data from either the same or yet another sensor unit 110. In some embodiments, the server 135 may “pull” the data streams, e.g., by querying the sensor units 110 and/or the local computing devices 115, 120. In some embodiments, the data streams may be “pushed” from the sensor units 110 and/or the local computing devices 115, 120 to the server 135. For example, the sensor units 110 and/or the local computing devices 115, 120 may be configured to transmit data as it is generated by or entered into that device. In some instances, the sensor units 110 and/or the local computing devices 115, 120 may periodically transmit data (e.g., as a block of data or as one or more data points).

The server 135 may include a database (e.g., in memory) containing physiological and/or location data received from the sensor units 110 and/or the local computing devices 115, 120. Additionally, as described in further detail herein, software (e.g., stored in memory) may be executed on a processor of the server 135. Such software (executed on the processor) may be operable to cause the server 135 to monitor, process, summarize, present, and/or send a signal associated with physiological and/or location data.

Although the server 135 and the remote computing device 145 are shown and described as separate computing devices, in some embodiments, the remote computing device 145 may perform the functions of the server 135 such that a separate server 135 may not be necessary. In such an embodiment, the remote computing device 145 may receive physiological and/or location data streams from the sensor units 110 and/or the local computing devices 115, 120, process the received data, and display the processed data on a single visual display, such as a map or image.

Additionally, although the remote computing device 145 and the local computing devices 115, 120 are shown and described as separate computing devices, in some embodiments, the remote computing device 145 may perform the functions of the local computing devices 115, 120 such that a separate local computing device 115, 120 may not be necessary. In such an embodiment, the third-party user (e.g., a nurse or a coach) may manually enter the user's physiological and/or location data (e.g., the user's body temperature, location on a sports field or track, etc.) directly into the remote computing device 145.

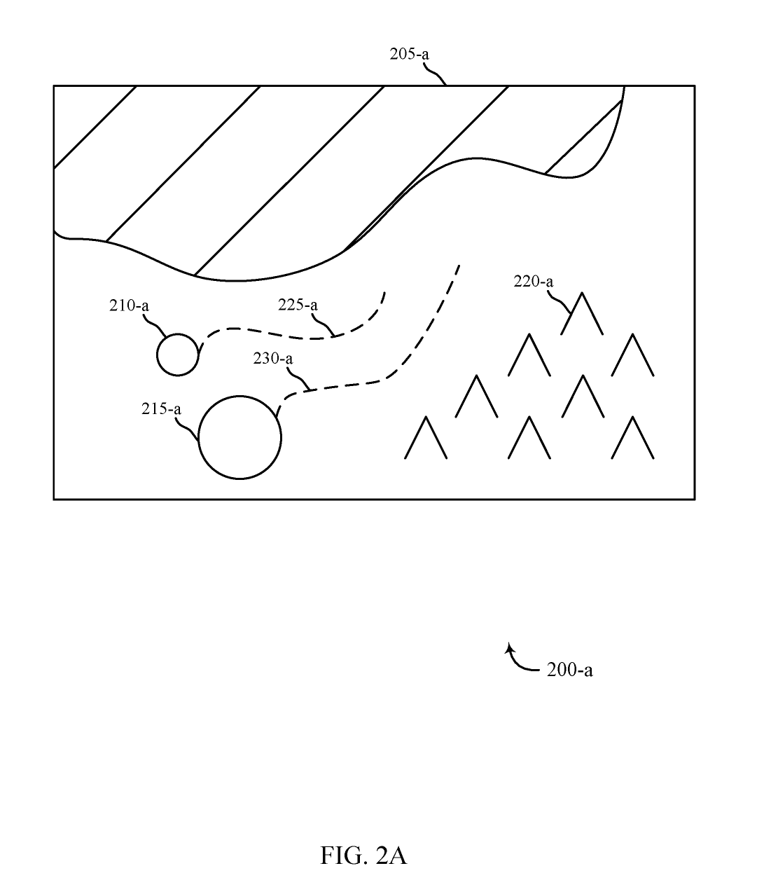

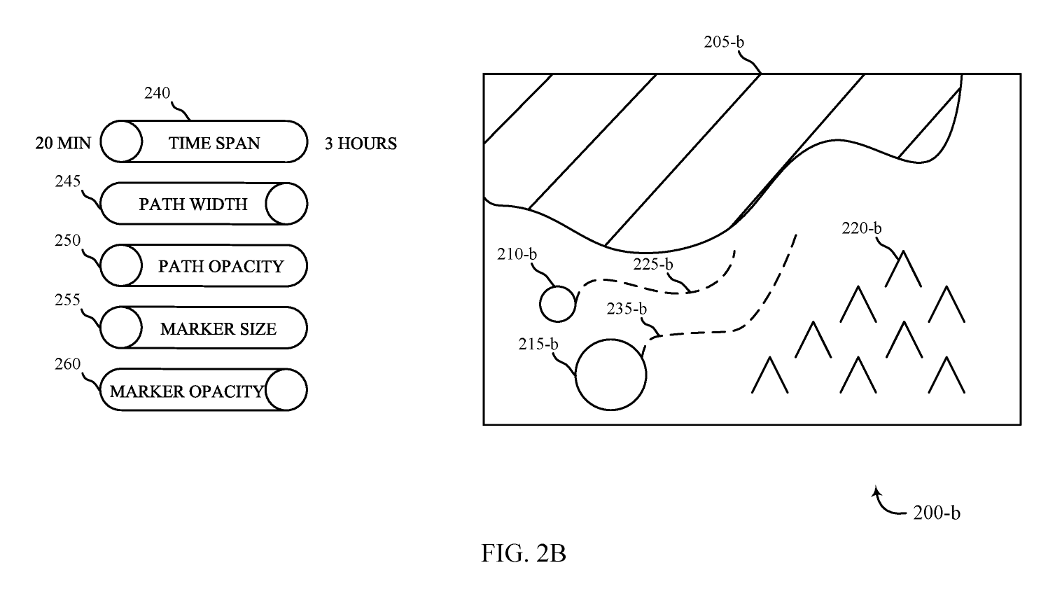

FIG. 2A is an example user interface displaying physiological data and location data on a same visual representation for one or more users. In the example illustration 200-a, the current location for each of two users, a first user 210-a and a second user 215-a, is illustrated overlaid on an image of a map 205-a. The map 205-a may be shown as a satellite view of the users' 210-a, 215-a current locations, including various topographical elements 220-a in some examples, or in other examples may be a “street view” or other view showing only roads, trails, and other marked courses. In still other examples, the map 205-a may be shown as a hybrid view, showing both topography and manmade features.

In illustration 200-a, first user 210-a and second user 215-a are shown as dots on the map to indicate each user's current position. The users' current positions may be updated continuously to show the users' positions in real-time, or alternatively may be updated at predetermined intervals or on demand. Additionally, the users' “snail trails,” showing their previous positions on the map 205-a over a monitored period of time, may be illustrated as dotted line 225-a for the first user 210-a, and dotted line 230-a for the second user 215-a. As the users move across the terrain of map 205-a, their snail trails 225-a, 230-a may extend to show the entirety of their paths traveled over the monitored training period. In other examples, the users' current positions may be shown using dots, shapes, or other identifiers, and in some examples, their snail trails may not be included.

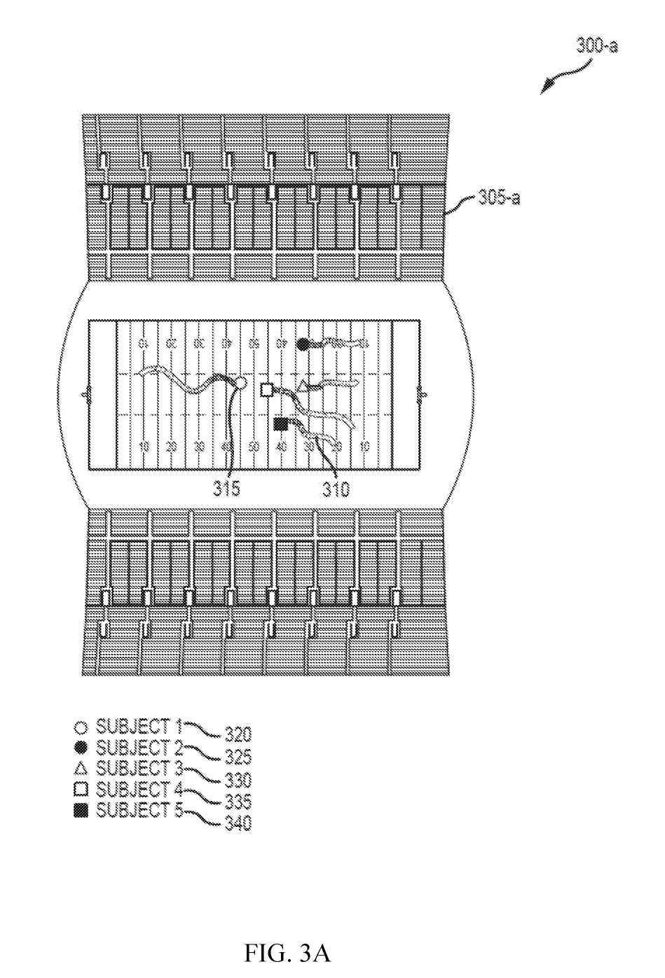

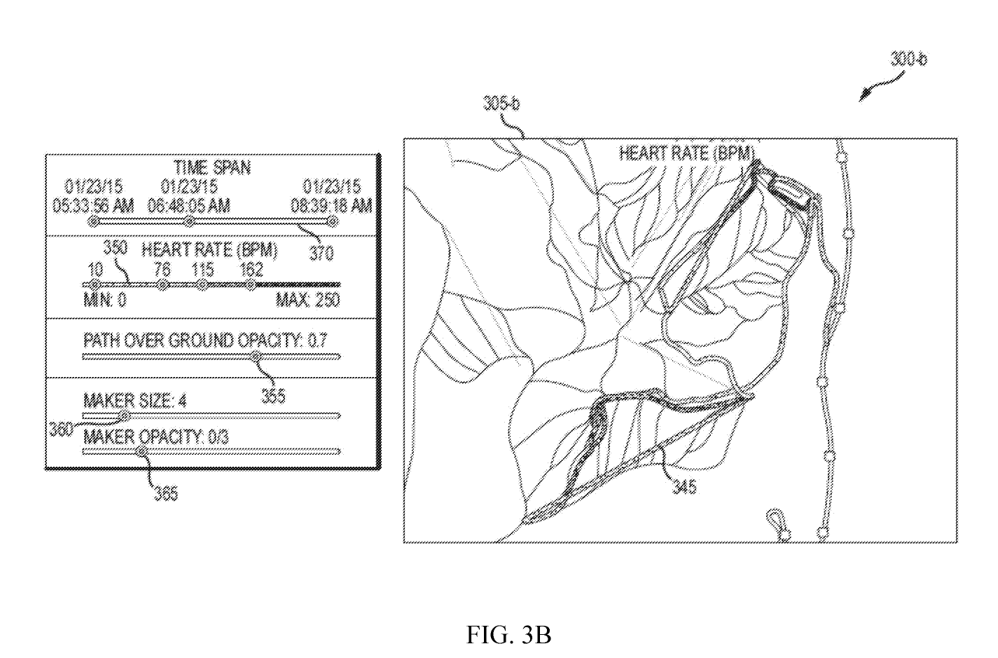

In addition to illustrating user positions, the dots representing the first user 210-a and second user 215-a may be indicative of a monitored physiological parameter for each user. For example, as shown in illustration 200-a, the dot representing the first user 210-a is shown smaller than the dot illustrating second user 215-a. The size of each dot may be indicative of, for example, each user's heart rate, where the dot may increase in diameter as the user's heart rate increases, and may decrease in diameter as the user's heart rate decreases. The relative sizes of each dot representing the first user 210-a and second user 215-a may accordingly be utilized to compare the physiological fitness or efficacy of each user's training at a glance, without the need to look up separately displayed numerical data. For example, if first user 210-a and second user 215-a are running on a trail together, and map 205-a demonstrates that the two users are side-by-side on the trail and therefore running at the same pace, the relative size of the dots representing each of the users may be utilized to indicate that the first user 210-a has a lower heart rate, and is therefore working less hard (or more efficiently), than second user 215-a, because the second user 215-a has a larger diameter dot and therefore a higher heart rate. In other examples, the diameters of the dots indicating the locations of the users may be indicative of various other monitored physiological or environmental parameters, such as respiration rate, body temperature, blood oxygen level, altitude, or the like. Additionally or alternatively, user locations and physiological parameters may be demonstrated by various other visual identifiers, such as colors, shapes, “heat glow,” and the like, as discussed in further detail below with respect to FIGS. 3A and 3B. User physiological data may be updated continuously, such that the size, color, shape, glow, or the like, representing the user's location and physiological parameters may be continuously updated to show real-time user physiological data. In other examples, the size, etc. of the visual representation of the user's location and physiological data may be updated at predetermined temporal intervals, or may be updated whenever the user's physiological parameters enter a new training zone; for example, where a user's heart rate passes from a “yellow training zone” indicating mid-level exertion into a “red training zone,” indicating rigorous exertion.

FIG. 2B is similarly an illustration 200-b of an example user interface for simultaneously displaying location data and physiological data for a plurality of users in a single illustration. Just as is shown in FIG. 2A, FIG. 2B illustrates a first user 210-b and a second user 215-b, the locations for whom are shown by dots, and the previous locations for whom are illustrated with dotted snail trail lines. In addition, FIG. 2B illustrates topographical features 220-b demonstrative of the location and terrain in which the first and second users are training.

In illustration 200-b, the user interface also includes a series of toggle switches configured to allow for user manipulation of display features. Various toggle switches may be included in the user interface; in illustration 200-b, a user may adjust any one or more of time span monitored 240, path width 245, path opacity 250, marker size 255, or marker opacity 260. For example, a user may choose to display only the last 45 minutes of monitored user location and physiological parameters, and may manipulate toggle switch 240 accordingly. Other additional or alternative display features may also be manipulated by a user.

FIG. 3A is an illustration 300 of an example user interface for simultaneously displaying location data and physiological data for a plurality of users. Where a point is used to indicate a user's current or more recent position, the physiological data displayed with the point, for example as a color, may be continuously updated to reflect changes in user physiological data. In other examples, the user's current condition may be illustrated as a shape, such as a circle, square, or triangle, and his physiological data may be displayed as updates to the shape and/or to shading associated with the shape. Thus, over time, a point or shape indicating a user's position on a map may move on the map to show the user's updated position, and may also change in color (or size, shape, glow, etc.) to show the user's updated physiological data. In other examples, a line or trail may be used to represent a user's position over time. Where a line or trail is used, changes in a user's physiological status may be shown over time as a series of points or segments of varying colors, trail width, or glow along the line or trail. In the illustrated example, the degree to which the line or trail is stippled may be used to indicate changes in the user's physiological status. For example, a user going for a run may be illustrated as a line on a map of the city in which the user is running. The line may move with the user to indicate updates in the user's position and to provide a “snail trail” indicating where the user has been. The color or concentration of stipple of the line may also be updated to indicate changes in the user's physiological state. For example, for the first mile, the user's heart rate may be elevated to 85% of his maximum heart rate, and the snail trail for that corresponding mile may accordingly be shown in orange, or with a high concentration of stipple. Between the first and second miles, the user may speed up or climb a hill, such that his heart rate may increase to 93% of his maximum heart rate, illustrated as a red or maximally stippled section of the snail trail for that corresponding second mile. As the user's heart rate changes, the color or degree of stipple of his snail trail may correspondingly change for the applicable segment of time or distance. Where the user's physiological data is updated in real-time, the color or level of stipple of each segment of trail may indicate real-time updates in the user's heart rate or other monitored physiological parameter. Alternatively, where the user's physiological data is displayed at predetermined intervals, the color or degree of stipple of each segment of trail corresponding to each interval may represent the average heart rate, or other monitored physiological parameter, measured for the user over that interval of time. These color-coded or stippled points or trails may be useful in pinpointing problem areas and tracking improvements in user training regimens.

The variations in size, color, stipple, glow, opacity, or the like of the points, lines, or trails presented on the map or image to indicate user physiological data may be correlated to various predetermined training or monitoring zones. The training or monitoring zones may be selected to correspond to individual user physiological conditions. For example, a user may input that he has a maximum heart rate of 190 beats per minute, and a resting heart rate of 60 beats per minute, based upon his weight, age, and other individual physiological factors. A range of five training zones between these provided minimum and maximum heart rates may accordingly be derived; for example, zone 1 (blue or little to no stipple) may be indicative of a heart rate between 60-86 beats per minute (bpm), or roughly 30-45% of the user's maximum heart rate; zone 2 (green or minimal stipple) may represent a heart rate between 87-113 bpm, or about 46-59% of the user's maximum heart rate; zone 3 (yellow or more concentrated stipple) may represent a heart rate between 114-140 bpm, or about 60-74% of the user's maximum heart rate; zone 4 (orange or high levels of stipple) may represent a heart rate between 141-167 bpm, or about 75-88% of the user's maximum heart rate; and zone 5 (red or maximized stipple) may represent a heart rate between 168-190 bpm, or about 89-100% of the user's maximum heart rate. Although discussed in this example as equal segments, the five training zones may be, in other examples, unevenly divided according to individual users' training goals and physiological parameters. For example, for a well-conditioned athlete, the “red” or maximally stippled zone may comprise only the top 5% of the athlete's maximum heart rate, while the “blue” or minimally stippled zone may comprise the bottom 25% of the athlete's maximum heart rate. Thus, by quickly reviewing the map of his run, a user may easily determine the points along his route at which his heart rate exceeded, met, or failed to meet his individual training goals.

In some examples, multiple users may be monitored concurrently on the same map or image. This may be particularly useful, for example, for sports teams. For example, a coach may be able to view the current and relative positions of each of his players, overlaid on an image of a football field, while simultaneously monitoring each player's physical status. In this way, a coach may compare, for example, two or more players' relative speeds and accelerations over a period of time by viewing a snail trail color-coded or stippled to illustrate player speed. As the players cover ground on the field, their respective snail trails may change in color or degree of stipple to indicate an increase in speed, and the coach may be able to view the comparative speeds of each player at the same moment in time to determine which player accelerated in the shortest period of time. This may provide helpful training data, compiled in a single, user-friendly visual representation.

In addition to applications in sports, the ability to monitor user position and physiological parameters may also be useful in military applications. For example, a unit leader may be able to readily view the current position and physiological status of each of his troops, and may quickly determine those who are in danger, for example due to an unusually high or unusually low respiration rate. The unit leader may also be able to quickly identify healthy troops positioned nearby the at-risk soldier such that the healthy troops may provide assistance to the at-risk soldier in the field.

In some embodiments, the “heat glow” of a point indicating a user's position may be used to indicate a period of time spent by the user at a particular position. For example, a soccer player may not be in motion for the entire duration of his practice or game, but may instead spend discrete periods of time stationary on the field. As the player stands in one spot for an increasing period of time, a colored “glow” may increase in size or radius, or may change in color or level of stippling, around the point indicating the player's position. In this way, a coach may be able to monitor excessive periods of immobility for his players both in real-time and upon review after the game or practice has ended. Similarly, a military unit leader may be able to send help for a troop who has been immobile for a troubling period of time.

In addition to directly measurable physiological parameters, such as heart rate and speed, in some examples the points, lines, or trails indicating a user's position on a map or image may also be utilized to display physiological and/or mechanical intensity for a user. For example, physiological intensity may be calculated based on a percentage of the user's maximum heart rate, and may be correlated to a series of training zones and corresponding shapes, colors, stippling, opacities, etc. The physiological intensity for the user may then be monitored continuously or at discrete intervals, and may be recorded as predetermined periods of time spent in each zone. For example, the measured physiological intensity for each second may be monitored over the course of one minute, and the average or maintained physiological intensity for the one minute may be correlated to a training zone. The physiological intensity may be summed over time to quantify a user's activity. This may be useful to compare physiological intensity between two or more users. For example, by monitoring the physiological intensity for two runners running at the same pace and for the same distance, it may be determined that one runner is working harder (at a higher physiological intensity), and is therefore in lesser shape or is running less efficiently, than a second runner who is working at a lower physiological intensity and is obtaining the same pace and distance results.

Mechanical intensity may similarly be used to determine intensity of user movement. For example, the determined mechanical intensity for a basketball player walking on the court may be 1 g (where a “g” is equal to the force of acceleration due to gravity near the Earth's surface), while the same basketball player running down the court, cutting, or jumping, may achieve a mechanical intensity of up to 7 g. The mechanical intensity training zone scale may accordingly be measured from 1 g to 10 g, with five discreet zones along that scale, to quantify mechanical intensity for that player. For each second, the maximum g-force calculated for that second may be correlated to an intensity zone. The measured intensities over time may be summed in order to quantify the total amount of motion and/or bodily impact experienced by the user over the course of the training or game. The mechanical intensity scale may be customized according to individual user physiological factors and the type of activity being performed. For example, speed skaters glide over ice and therefore should experience very little variation in mechanical intensity; accordingly, the training scale for a speed skater may range only from 0.2-1.5 g, such that mechanical load and intensity for individual skaters, and accordingly efficiency and smoothness, may be monitored with precision.

In some examples, the visual display on which user location and physiological parameter data are illustrated may include a geo-tagged image. For example, an image of a football field, soccer field, baseball field, rugby pitch, track, lacrosse field, ice hockey rink, field hockey field, and plain green field with map scale may be displayed as a template, and may be geo-tagged with latitude and longitude positions.

In the illustrated example 300-a, the user interface is shown as an aerial view of a football stadium 305-a, viewable for example on a dedicated application on a smartphone, tablet, or personal computer, or alternatively or additionally viewable on a webpage on a remote computing device.

Collected location data and physiological data for the plurality of users may be displayed on a same visual representation for each of the plurality of users. For example, as shown on the aerial view of the stadium 305-a, five users 320, 325, 330, 335, 340, in this example football players, are represented by five various shapes and corresponding lines or “snail trails.” For example, white circle 315 is demonstrative of the real-time location of a first player 320, while snail trail 310 is indicative of the path traveled by a fifth player 340 over a monitored period of time. Varying color or shape may be representative of the identity of the individual player being monitored, while the varying color or concentration of stippling of each snail trail may indicate a changing physiological parameter monitored over a predetermined period of time. For example, in the illustrated example, the snail trail 310 may progress from blue, to green, to yellow, to orange, to red over the monitored time period, or may change from no stippling, to limited stippling, to a medium level of stippling, to more concentrated stippling, to maximized stippling, indicating that the monitored player has increased his speed, for example, across five predetermined training zones. The blue and green (or no stippling and limited stippling) zones may indicate slower paces, while the yellow (or medium level of stippling) zone may indicative of a moderate pace, and the orange and red (or more concentrated and maximized stippling) zones may be indicative of increased speeds. Thus, the illustration 300-a may provide a visual representation of five football players 320, 325, 330, 335, 340 running towards each other on a football field, where a coach may monitor the speed at which each player accelerated towards the others.

In some examples, a coach may review the comparative speeds of each of the five players 320, 325, 330, 335, 340 by manipulating the visual representations on the field. For example, by selecting a particular time during the training period, or by dragging the shapes representing each player backward along their snail trails, the coach may be able to view the relative positions and speeds of each player at discrete times during the training or play. Thus, for example, a coach may be able to visualize that, at five seconds after the play had started, player one 320 had reached a speed represented by the third, yellow (or medium stippled) training zone, while player two 325 was still only in the second, green (or limited stippling) training zone. This may indicate to the coach that player one 320 has a more powerful or efficient acceleration that that of player two 325.

Although discussed with respect to speed and acceleration, snail trails 310 may also be representative of any one or more other physiological parameters, such as heart rate, respiration rate, body temperature, and the like. In some examples, physiological data over time may be represented as a series of dots, rather than snail trails. In other examples, physiological data may be represented by a heat radius or “glow” around each dot 315 representing the monitored users. In addition, although illustration 300-a is shown as a football field, in other examples, the visual representation of the monitored users' location and physiological data may be displayed on any other suitable image.

FIG. 3B is an illustration 300-b of an alternate visual representation of one or more users' physiological parameters and locations over time. The illustration 300-b may be an interactive user interface, and may be viewable, for example, on a dedicated application on a user's smartphone or personal computer, or on a body-worn display device.

In the example shown in illustration 300-b, the visual representation is displayed on a map of a ski area 305-b. In this example, the location and physiological data for a single user is monitored over the course of the user's day at the ski area. For example, snail trail 345 depicts the user's movement around the ski resort. As the user progresses between physically active periods of skiing, and more restful periods of sitting on the chairlift, the user's heart rate, and accordingly the visual display thereof, varies along the snail trail.

In illustration 300-b, a user viewing the visual representation may vary certain parameters in order to customize the visual representation to the user being monitored. For example, the training zones representing different ranges of a monitored physiological parameter may be varied as shown by reference numeral 350 in order to align with individual user physiological parameters. Where the monitored user is more physically fit, the fifth, red (or maximally stippled) training zone representing the highest heart rate range may be reduced, for example to encompass only heart rates between 190-250 beats per minute (bpm), while for a less active monitored user, the fifth training zone may be larger, as shown, ranging from 162-250 bpm.

Other visual representation parameters, such as ground opacity 355, marker size 360, and marker opacity 365 may also be manipulated by the user in order to convey the desired information regarding the monitored user's training.

In some examples, two or more physiological parameters may be illustrated by the single visual representation. For example, in illustration 300-b, while the snail trail color or degree of stippling may vary to represent changes in the user's heart rate, the width or “glow” of the snail trail may similarly change based on a period of time spent by the user in a particular area. For example, the snail trail indicating a user's time spent skiing down a slope may be more narrow, indicating that the user quickly traversed that area, while the snail trail indicating the user's time spent on a slower-moving chairlift, or pausing at the base of the mountain, may be thicker or have a larger heat “glow,” indicating a comparatively greater period of time spent in that location. In this way, the user's speed may be monitored, in addition to his location and heart rate, or other monitored physiological parameter.

In addition, the period of time monitored may be varied based on user preferences, as indicated by reference numeral 370. For example, a user may wish only to view the location and physiological parameters for the monitored user between 5:33 am and 6:48 am, or may instead wish to view all data gathered between 5:33 am and 8:39 am. By manipulating the monitored scale, the user may view only that period which is of interest.

Over the monitored period of time, the user's location and physiological parameters may be monitored, updated, and/or illustrated continuously, for example each second, or in some examples may be monitored, updated, and/or illustrated at predetermined intervals. In some examples, the physiological parameters displayed as, for example, varying colors, degrees of stippling, or heat glows may represent an average of the monitored physiological data over a predetermined period of time. The location and physiological data for the user may be monitored in real-time in some examples, or may be reviewed after the fact in other examples.

Although shown in illustration 300-b as data for a single user, in other embodiments, a plurality of users may be monitored on a single illustration, as discussed in more detail above with respect to FIG. 3A.

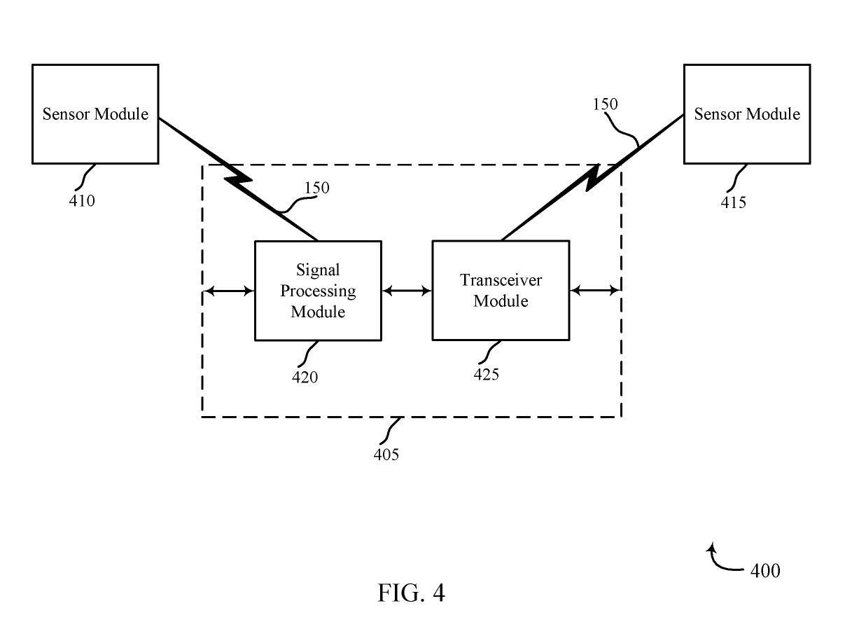

FIG. 4 shows a block diagram 400 that includes apparatus 405, which may be an example of one or more aspects of the sensor unit 110, third-party sensor 130, local computing devices 115, 120, and/or remote computing device 145 (of FIG. 1) for use in physiological and/or location monitoring, in accordance with various aspects of the present disclosure. In some examples, the apparatus 405 may include a signal processing module 420 and a transceiver module 425. In some examples, one or more sensor modules 410, 415 may be positioned externally to apparatus 405 and may communicate with apparatus 405 via wireless links 150, or in other examples the one or more sensor modules 410, 415 may be components of apparatus 405. Each of these components may be in communication with each other.

The components of the apparatus 405 may, individually or collectively, be implemented using one or more application-specific integrated circuits (ASICs) adapted to perform some or all of the applicable functions in hardware. Alternatively, the functions may be performed by one or more other processing units (or cores), on one or more integrated circuits. In other examples, other types of integrated circuits may be used (e.g., Structured/Platform ASICs, Field Programmable Gate Arrays (FPGAs), and other Semi-Custom ICs), which may be programmed in any manner known in the art. The functions of each unit may also be implemented, in whole or in part, with instructions embodied in a memory, formatted to be executed by one or more general or application-specific processors.

In some examples, the transceiver module 425 may be operable to receive data streams from the sensor units 110 and/or sensor modules 410, 415, as well as to send and/or receive other signals between the sensor units 110 and either the local computing devices 115, 120 or the remote computing device 145 via the network 125 and server 135. The transceiver module 425 may include wired and/or wireless connectors. For example, in some embodiments, sensor units 110 may be portions of a wired or wireless sensor network, and may communicate with the local computing devices 115, 120 and/or remote computing device 145 using either a wired or wireless network. The transceiver module 425 may be a wireless network interface controller (“NIC”), Bluetooth® controller, IR communication controller, ZigBee® controller and/or the like.

In some examples, the signal processing module 420 may include circuitry, logic, hardware and/or software for processing the data streams received from the sensing units 110 and/or sensor modules 410, 415. The signal processing module 420 may include filters, analog-to-digital converters and other digital signal processing units. Data processed by the signal processing module 420 may be stored in a buffer, for example.

Sensor modules 410, 415 may comprise any combination of physiological and/or location sensing components, including, for example, heart rate monitors, respiration monitors, blood pressure monitors, pulse monitors, orientation monitors, accelerometers, temperature monitors, global positioning sensors, force monitors, and the like.

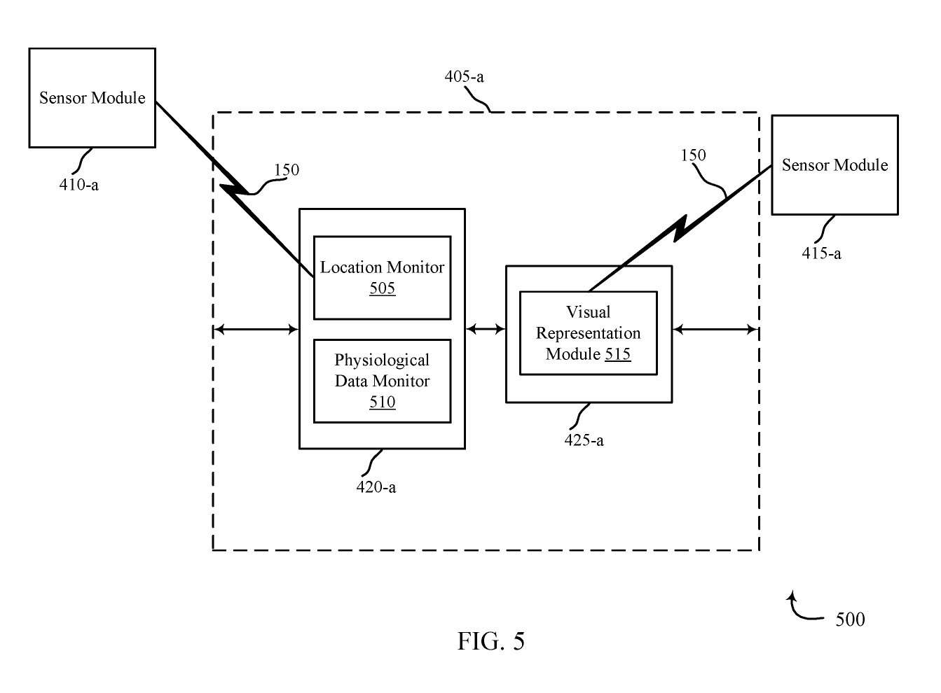

FIG. 5 shows a block diagram 500 that includes apparatus 405-a, which may be an example of apparatus 405 (of FIG. 4), in accordance with various aspects of the present disclosure. In some examples, the apparatus 405-a may include a signal processing module 420-a, a transceiver module 425-a, and one or more sensor modules 410-a, 415-a, which may be examples of the signal processing module 420, the transceiver module 425, and one or more sensor modules 410, 415 of FIG. 4. In some examples, one or more sensor modules 410-a, 415-a may be positioned outside of apparatus 405-a, while in other examples, one or more sensor modules 410-a, 415-a may be components of apparatus 405-a. In some examples, signal processing module 420-a may include one or more of a location monitor 505 and a physiological data monitor 510. In some examples, transceiver module 425-a may include a visual representation module 515. Additionally, while FIG. 5 illustrates a specific example, the functions performed by each of the modules 505, 510, and 515 may be combined or implemented in one or more other modules.

The location monitor 505 may be operable to detect a location of the user at predetermined intervals or continuously in real-time. For example, the location monitor 505 may receive a stream of location data from one or more sensor modules 410-a, 415-a.

The physiological data monitor 510 may be operable to detect various physiological parameters for the user, also at either predetermined intervals or continuously in real-time. For example, the physiological data monitor 510 may receive a stream of heart rate data from one sensor module 410-a, and may receive a stream of respiratory rate data from a second sensor module 415-a, or from the same sensor module 410-a. In some examples, the physiological data monitor 510 may collect a stream of physiological data and average the data over a predetermined period of time.

Each of the derived location of the user from location monitor 505 and physiological data for the user from physiological data monitor 510 may be communicated to visual representation module 515. Visual representation module 515 may be operable to collect the received location and physiological data, synchronize the data according to corresponding timestamps for each data stream, and derive a single visual display of the data, for example on a map or image. In some examples, the location may be displayed as a dot or point, line, or shape, for example on a map or an image of a sports field or other location. In some examples, one or more set of physiological data may be displayed simultaneously with the location data, for example by providing various colors, opacities, or heat radii of the dot, line, or shape. For example, a user's location on a map may be shown as a dot, and his current body temperature may be shown as a color of the dot, where the color may correspond to a temperature range or zone. As visual representation module 515 receives updated location and physiological data from location monitor 505 and physiological data monitor 510, the visual representation of that data may be updated. For example, the dot may move on the map to show a new location of the user, and/or the color of the dot may change to represent a different body temperature zone.

In some examples, the visual representation derived by visual representation module 515 may be displayed at apparatus 405-a. For example, apparatus 405-a may be a smartphone or other personal computing device having a display screen, and the visual representation may be displayed on the screen, for example as part of a dedicated application. In other examples, the visual representation derived by visual representation module 515 may be communicated to a remote computing device 145 for display. In some examples, the visual representation may be accessed as part of a dedicated application, while in other examples the visual representation may be accessed via a website. In some examples, as discussed above with respect to FIGS. 2 and 3, the visual representation may be interactive.

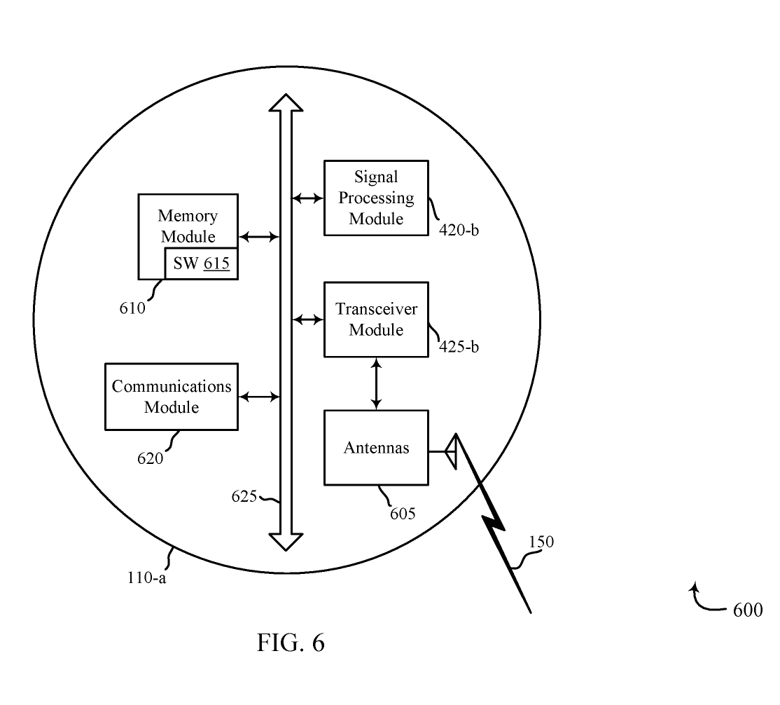

FIG. 6 shows a block diagram 600 of a sensor unit 110-a for use in remote physiological and location data monitoring, in accordance with various aspects of the present disclosure. The sensor unit 110-a may have various configurations. The sensor unit 110-a may, in some examples, have an internal power supply (not shown), such as a small battery, to facilitate mobile operation. In some examples, the sensor unit 110-a may be an example of one or more aspects of one of the sensor units 110 and/or apparatus 405, 405-a described with reference to FIGS. 1, 4 and/or 5. In some examples, the sensor unit 110-a may be an example of one or more aspects of one of the sensor modules 410, 415 or 410-a, 415-a described with reference to FIGS. 4 and/or 5. The sensor unit 110-a may be configured to implement at least some of the features and functions described with reference to FIGS. 1, 4 and/or 5.

The sensor unit 110-a may include a signal processing module 420-b, a transceiver module 425-b, a communications module 620, at least one antenna (represented by antennas 605), and/or a memory module 610. Each of these components may be in communication with each other, directly or indirectly, over one or more buses 625. The signal processing module 420-b and transceiver module 425-b may be examples of the signal processing module 420 and transceiver module 425, respectively, of FIG. 4.

The memory module 610 may include RAM and/or ROM. The memory module 610 may store computer-readable, computer-executable code (SW) 615 containing instructions that are configured to, when executed, cause the signal processing module 420-b to perform various functions described herein related to simultaneously displaying location data and physiological data. Alternatively, the code 615 may not be directly executable by the signal processing module 420-b but may be configured to cause the server 135 (of FIG. 1) (e.g., when compiled and executed) to perform various of the functions described herein.

The signal processing module 420-b may include an intelligent hardware device, e.g., a CPU, a microcontroller, an ASIC, etc. The signal processing module 420-b may process information received through the transceiver module 425-b or information to be sent to the transceiver module 425-b for transmission through the antenna 605. The signal processing module 420-b may handle various aspects of signal processing as well as deriving a visual representation of the received physiological and location data.

The transceiver module 425-b may include a modem configured to modulate packets and provide the modulated packets to the antennas 605 for transmission, and to demodulate packets received from the antennas 605. The transceiver module 425-b may, in some examples, be implemented as one or more transmitter modules and one or more separate receiver modules. The transceiver module 425-b may support visual representation communications. The transceiver module 425-b may be configured to communicate bi-directionally, via the antennas 605 and communication link 150, with, for example, local computing devices 115, 120 and/or the remote computing device 145 (via network 125 and server 135 of FIG. 1). Communications through the transceiver module 425-b may be coordinated, at least in part, by the communications module 620. While the sensor unit 110-a may include a single antenna 605, there may be examples in which the sensor unit 110-a may include multiple antennas 605.

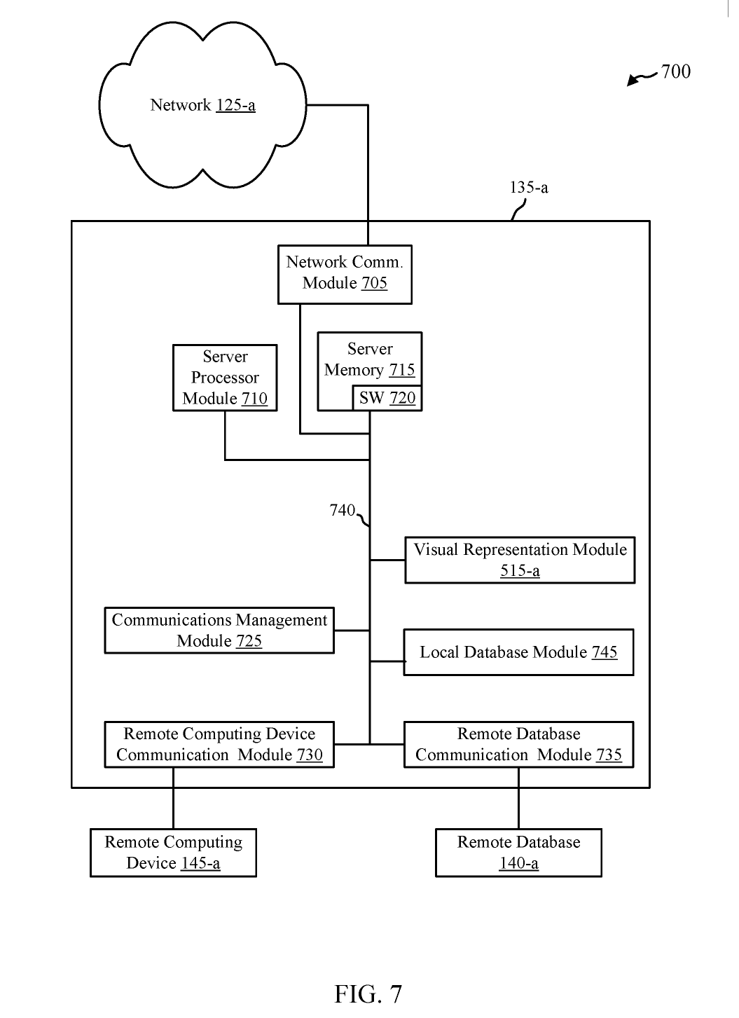

FIG. 7 shows a block diagram 700 of a server 135-a for use in simultaneously displaying location data and physiological data for one or more monitored users, in accordance with various aspects of the present disclosure. In some examples, the server 135-a may be an example of aspects of the server 135 described with reference to FIG. 1. In other examples, the server 135-a may be implemented in either the local computing devices 115, 120 or the remote computing device 145 of FIG. 1. The server 135-a may be configured to implement or facilitate at least some of the features and functions described with reference to the server 135, the local computing devices 115, 120 and/or the remote computing device 145 of FIG. 1.

The server 135-a may include a server processor module 710, a server memory module 715, a local database module 745, and/or a communications management module 725. The server 135-a may also include one or more of a network communication module 705, a remote computing device communication module 730, and/or a remote database communication module 735. Each of these components may be in communication with each other, directly or indirectly, over one or more buses 740.

The server memory module 715 may include RAM and/or ROM. The server memory module 715 may store computer-readable, computer-executable code (SW) 720 containing instructions that are configured to, when executed, cause the server processor module 710 to perform various functions described herein related to displaying location data and physiological data simultaneously for one or more monitored user. Alternatively, the code 720 may not be directly executable by the server processor module 710 but may be configured to cause the server 135-a (e.g., when compiled and executed) to perform various of the functions described herein.

The server processor module 710 may include an intelligent hardware device, e.g., a central processing unit (CPU), a microcontroller, an ASIC, etc. The server processor module 710 may process information received through the one or more communication modules 705, 730, 735. The server processor module 710 may also process information to be sent to the one or more communication modules 705, 730, 735 for transmission. Communications received at or transmitted from the network communication module 705 may be received from or transmitted to sensor units 110, local computing devices 115, 120, or third-party sensors 130 via network 125-a, which may be an example of the network 125 described in relation to FIG. 1. Communications received at or transmitted from the remote computing device communication module 730 may be received from or transmitted to remote computing device 145-a, which may be an example of the remote computing device 145 described in relation to FIG. 1. Communications received at or transmitted from the remote database communication module 735 may be received from or transmitted to remote database 140-a, which may be an example of the remote database 140 described in relation to FIG. 1. Additionally, a local database may be accessed and stored at the server 135-a. The local database module 745 may be used to access and manage the local database, which may include data received from the sensor units 110, the local computing devices 115, 120, the remote computing devices 145 or the third-party sensors 130 (of FIG. 1).

The server 135-a may also include a visual representation module 515-a, which may be an example of the visual representation module 515 of apparatus 405-a described in relation to FIG. 5. The visual representation module 515-a may perform some or all of the features and functions described in relation to the visual representation module 515, including processing physiological data and location data for one or more user received from the location monitor 505 and physiological data monitor 510, respectively, as described in relation to FIG. 5, in order to compile and simultaneously display the location and physiological data as a single visual representation.

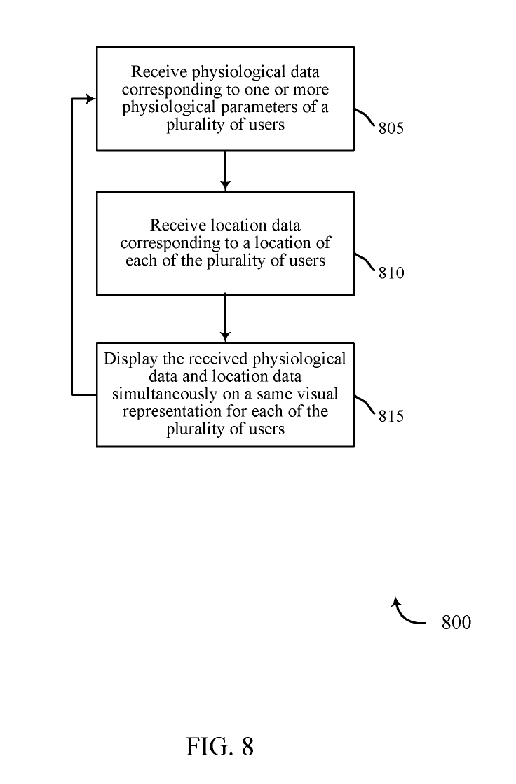

FIG. 8 is a flow chart illustrating an example of a method 800 for simultaneously displaying location data and physiological data for one or more users, in accordance with various aspects of the present disclosure. For clarity, the method 800 is described below with reference to aspects of one or more of the local computing devices 115, 120, remote computing device 145, and/or server 135 described with reference to FIGS. 1, and/or 7, or aspects of one or more of the apparatus 405, 405-a described with reference to FIGS. 4 and/or 5. In some examples, a local computing device, remote computing device or server such as one of the local computing devices 115, 120, remote computing device 145, server 135 and/or an apparatus such as one of the apparatuses 405, 405-a may execute one or more sets of codes to control the functional elements of the local computing device, remote computing device, server or apparatus to perform the functions described below.

At block 805, the method 800 may include receiving physiological data corresponding to one or more physiological parameters of a plurality of users. The plurality of users may be wearing, holding, or otherwise associated with one or more sensor units, each of which may be operable to detect one or more physiological parameters of each of the plurality of users. For example, the one or more sensor units may detect any of user heart rate, respiration rate, body temperature, mechanical intensity, physiological intensity, training intensity, speed, distance traveled, time spent in a position, or altitude, or a combination thereof.

At block 810, the method 800 may include receiving location data corresponding to a location of each of the plurality of users. As previously discussed, the one or more sensor units may be operable to receive global positioning data. Alternatively or in addition, each of the plurality of users' location may be tracked by a third-party sensor, or may be manually inputted by a third-party user.

Each of the received physiological data and received location data for the plurality of users may be monitored continuously or at predetermined intervals. The physiological data and location data received may be time-stamped, such that the physiological data and location data may be properly correlated in the visual display, as discussed in more detail below.

At block 815, the method 800 may include displaying the received physiological data and location data simultaneously on a same visual representation for each of the plurality of users. In some examples, the physiological data and location data may be displayed on the same visual representation on any of a map or an image. For example, the data may be displayed on an image of a football field or other sports arena. The same visual representation may include a combination of two or more of a dot or point, a line, a color, a heat radius, or a shape. For example, the position of a dot on a map may be indicative of the location of a user, while the color of that dot, or the heat radius around the dot, may be representative of one or more physiological parameters of the user, such as a speed or heart rate of the user.

The method 800 may proceed continuously from step 815 back to step 805 and step 810, such that physiological data and location data may be continuously received, or received at predetermined intervals, and such that the visual representation of the received physiological data and location data may be continuously or periodically updated to represent the most recent or real-time data for each of the plurality of users. For example, where the location of each of the plurality of users is represented by a plurality of dots, each of the plurality of dots may move on the map to correspond with the updated location data. Similarly, the color or shape of each of the plurality of dots may be updated to correspond with updated physiological data corresponding to one or more physiological parameters of each of the plurality of users.

In some embodiments, the operations at blocks 805, 810, or 815 may be performed using the visual representation module 515 described with reference to FIGS. 5 and/or 7. Nevertheless, it should be noted that the method 800 is just one implementation and that the operations of the method 800 may be rearranged or otherwise modified such that other implementations are possible.

The above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, the methods described may be performed in an order different from that described, and various steps may be added, omitted, or combined. Also, features described with respect to certain embodiments may be combined in other embodiments.

The detailed description set forth above in connection with the appended drawings describes exemplary embodiments and does not represent the only embodiments that may be implemented or that are within the scope of the claims. The term “exemplary” used throughout this description means “serving as an example, instance, or illustration,” and not “preferred” or “advantageous over other embodiments.” The detailed description includes specific details for the purpose of providing an understanding of the described techniques. These techniques, however, may be practiced without these specific details. In some instances, well-known structures and devices are shown in block diagram form in order to avoid obscuring the concepts of the described embodiments.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration. A processor may in some cases be in electronic communication with a memory, where the memory stores instructions that are executable by the processor.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope and spirit of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items indicates a disjunctive list such that, for example, a list of “at least one of A, B, or C” means A or B or C or AB or AC or BC or ABC (i.e., A and B and C).

A computer program product or computer-readable medium both include a computer-readable storage medium and communication medium, including any mediums that facilitates transfer of a computer program from one place to another. A storage medium may be any medium that may be accessed by a general purpose or special purpose computer. By way of example, and not limitation, computer-readable medium may comprise RAM, ROM, EEPROM, CD-ROM or other optical disk storage, magnetic disk storage or other magnetic storage devices, or any other medium that may be used to carry or store desired computer-readable program code in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote light source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk and blu-ray disc where disks usually reproduce data magnetically, while discs reproduce data optically with lasers. Combinations of the above are also included within the scope of computer-readable media.

The previous description of the disclosure is provided to enable a user skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” or “exemplary” indicates an example or instance and does not imply or require any preference for the noted example. Thus, the disclosure is not to be limited to the examples and designs described herein but is to be accorded the widest scope consistent with the principles and novel features disclosed herein.

1. A method for simultaneously displaying location data and physiological data for a plurality of users, comprising:
receiving physiological data corresponding to one or more physiological parameters of each of the plurality of users;
receiving location data corresponding to a location of each of the plurality of users; and
displaying the received physiological data and location data simultaneously on a same visual representation for each of the plurality of users.

2. The method of claim 1, further comprising:
displaying the physiological data and the location data on the same visual representation on a map or image.

3. The method of claim 1, further comprising:
indicating the physiological data and the location data on the same visual representation through a combination of two or more of a dot, a line, a color, a heat radius, or a shape.

4. The method of claim 1, further comprising:
continuously updating at least a portion of the visual representation for each of the plurality of users based at least in part on one or more of received physiological data and location data.

5. The method of claim 4, wherein continuously updating at least a portion of the visual representation comprises:
altering a shape of the visual representation, altering a color of the visual representation, altering a heat radius of the visual representation, altering a position of the visual representation, or altering an opacity of the visual representation, or a combination thereof.

6. The method of claim 1, wherein displaying the received physiological data and location data comprises:
displaying the received physiological data and location data in real-time.

7. The method of claim 1, wherein displaying the received physiological data and location data comprises:
displaying the received physiological data and location data for a predetermined period of time.

8. The method of claim 7, wherein at least one of the received physiological data and location data is displayed as an average over the predetermined period of time.

9. The method of claim 1, wherein the one or more physiological parameters of the plurality of users comprise a heart rate, a respiration rate, a body temperature, a mechanical intensity, a physiological intensity, a training intensity, a speed, a distance traveled, a time spent in a position, or an altitude, or a combination thereof.

10. The method of claim 1, further comprising:
associating the received physiological data with a plurality of predetermined training zones.

11. The method of claim 10, wherein each of the plurality of predetermined training zones is associated with a respective shape, size, opacity, heat radius, or color.

12. The method of claim 10, wherein the plurality of predetermined training zones are determined based at least in part on individual physiological parameters of each of the plurality of users, or individual training goals of each of the plurality users, or a combination thereof.

13. A system for simultaneously displaying location data and physiological data for a plurality users, comprising:
a transceiver configured to receive physiological data corresponding to one or more physiological parameters of each of the plurality of users and location data corresponding to a location of each of the plurality of users from one or more sensors; and
a processor configured to simultaneously display the received physiological data and location data on a same visual representation for each of the plurality of users.

14. The system of claim 13, wherein the processor is further configured to:
display the physiological data and the location data on the same visual representation on a map or an image.

15. The system of claim 13, wherein the processor is further configured to:
indicate the physiological data and the location data on the same visual representation through a combination of two or more of a dot, a line, a color, a heat radius, or a shape.

16. The system of claim 13, wherein the processor is further configured to:
continuously update at least a portion of the visual representation for each of the plurality of users based at least in part on one or more of the received physiological data and received location data.

17. The system of claim 16, wherein the processor is configured to continuously update a portion of the visual representation by:
altering a shape of the visual representation, altering a color of the visual representation, altering a heat radius of the visual representation, altering a position of the visual representation, or altering an opacity of the visual representation, or a combination thereof.

18. The system of claim 13, wherein the one or more physiological parameters of the plurality of users comprise a heart rate, a respiration rate, a body temperature, a mechanical intensity, a physiological intensity, a training intensity, a speed, a distance traveled, a time spent in a position, or an altitude, or a combination thereof.

19. The system of claim 13, wherein the received physiological data is associated with a plurality of predetermined training zones.

20. A non-transitory computer-readable medium storing computer-executable code, the code executable by a processor to:
receive physiological data corresponding to one or more physiological parameters of each of a plurality of users;
receive location data corresponding to a location of each of the plurality of users; and
display the received physiological data and location data simultaneously on a same visual representation for each of the plurality of users.
